(19) United States
(12) Patent Application Publication
(10) Pub. No.: US 2005/0095602 A1
(43) Pub. Date: May 5, 2005

(54) Microfluidic integrated microarrays for biological detection

(76) Inventors: Jason A.A. West, Castro Valley, CA (US); Timothy J. Shepodd, Livermore, CA (US); Stewart K. Griffiths, Livermore, CA (US); Ronald F. Renzi, Tracy, CA (US); Boyd J. Wiedenman, Alpharetta, GA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/701,097

(22) Filed: Nov. 4, 2003

(51) Int. Cl. C12Q 1/68; C12M 1/34

(52) U.S. Cl. 435/6; 435/287.2; 435/288.5

(57) ABSTRACT

Disclosed are microflulidic chips that include a plurality of vias; a functionalized porous polymer monolith capable of being in fluid communication with a via; a microarray capable of being in fluid communication with the functionalized porous polymer monolith; and an observation port through which at least one target disposed within the microarray is capable of being detected. The disclosed microfluidic chips contain microarrays that can be effectively coupled to functionalized porous polymer monoliths for capturing and concentrating sample nucleic acids. Also disclosed are microfluidic chips containing microarray probes having observation ports that enable the preparation of microarrays and the detection of targets. These microfluidic chips are capable of capturing and concentrating genetic material for the analysis and identification of biological organisms, such as so-called “threat genes” from chimeric bioweapons.

### STATEMENT OF GOVERNMENT INTEREST

This invention is made with Government support under contract no. DE-AC04-94AL85000 awarded by the U.S. Department of Energy to Sandia Corporation. The Government has certain rights in the invention.

### FIELD OF THE INVENTION

The present invention is related to the field of microfluidic devices. The present invention is also related to the field of biological detection.

### BACKGROUND OF THE INVENTION

Various scientific and patent publications are referred to herein. Each is incorporated by reference in its entirety.

Recent advances in miniaturization have led to the development of microfluidic systems that are designed, in part, to perform a multitude of chemical and physical processes on a micro-scale. Typical applications include analytical and medical instrumentation, industrial process control equipment, liquid and gas phase chromatography, and the detection of biological weapons. In this context, there is a need for devices that have fast response times to provide precise control over small flows as well as small volumes of fluid (liquid or gas) in microscale channels. In order to provide these advantages, flow control devices are typically integrated on microfluidic chips. The term “microfluidic chip” refers to a system or device having microchannels or microchambers that are generally fabricated on a substrate. The length scale of these microchannels is typically on the micron or submicron scale, i.e., having at least one cross-sectional dimension in the range from about 0.1 micron to about 500 microns. Examples of methods of fabricating microfluidic systems is known, as disclosed in U.S. Pat. No. 5,194,133 to Clark et al., U.S. Pat. No. 5,132,012 to Miura et al., U.S. Pat. No. 4,908,112 to Pace, U.S. Pat. No. 5,571,410 to Swedberg et al., U.S. Pat. No. 5,824,204 to Jerman, and U.S. Patent Application Pub. No. 2002/194,909 to Shepodd et al.

Recently, the development of DNA gene microarray or “microarray” technology capable of detecting thousands of genes in a single experimental test has rapidly advanced and become a widespread application technology. Two significant drawbacks to this technology in its current format are the long and tedious processing time for RNA/DNA sample preparation, often requiring up to four days. This problem is aggravated by the high sensitivity of RNA and DNA samples to degradation from ambient DNA and RNA nucleases. In order to tackle these inherent weaknesses in gene microarray analysis there is a need to develop microfluidic chips containing microarrays that can concentrate, bind and detect sample target genes using a single microfluidic chip. Such microfluidic chips could enable the development of portable devices that require reduced sample throughput time, decrease sample degradation, and small size.

Microfluidic chips that incorporate microarrays for carrying out genetic identification and analysis typically require that a biological sample containing nucleic acids (e.g., DNA and RNA) is captured and concentrated in a first step, which is then applied to the microarray in a second step. Typical methods for isolation and concentrating such targeted nucleic acids include gel-based separation processes, such as gel permeation chromatography, trapping on charged silica particles, and using specific or non specific complementary nucleotide sequences to facilitate hybridization of the targeted sequences. A number of problems are associated with using gel-based separation for capturing and concentrating sample nucleic acids. One problem with incorporating these processes on a microfluidic chip is the high pressures required to effect concentration or isolation typically exceed the operating pressures of a microfluidic chip. Another problem with the use of these materials is the containment of the trapping material typically require frits for preventing the material from exuding out of the isolation region under high pressure. The use of polymer gels in microfluidic chips is, accordingly, accompanied by low flow rates to maintain low operating pressures. Moreover, polymer gels typically separate analyte solutions based on analyte molecular size, and are generally non-specific to different molecules of similar size, such as nucleic acids. Accordingly, the utility of polymer gels is limited in nucleic acid identification and analysis in microfluidic devices. Thus, there is a need to provide microfluidic devices capable of capturing and concentrating biological samples for microarray analysis that overcome these problems.

Yu et al. describe a monolithic porous polymer for on-chip solid-phase extraction and preconcentration prepared by photoinitiated in situ polymerization within a microfluidic device. Analytical Chemistry, 73, No. 21, pp. 5088-5096 (2001). This reference discloses the preparation and use of monolithic materials for solid-phase extraction and preconcentration using a straight microchannel, but does not disclose the use of these monolithic materials for providing microfluidic chips with the capability of detecting and characterizing biological samples using microarrays.

### SUMMARY OF THE INVENTION

In overcoming the problems associated with providing a high throughput microfluidic chip capable of specifically capturing and concentrating nucleic acids for microarray analysis, the present invention provides, inter alia, microfluidic chips containing functionalized porous polymer monoliths for capturing and concentrating sample nucleic acids. In one aspect of the present invention, there are provided microfluidic chips that include a plurality of vias; a functionalized porous polymer monolith capable of being in fluid communication with a via; a microarray capable of being in fluid communication with the functionalized porous polymer monolith; and an observation port through which at least one target disposed within the microarray is capable of being detected. As will be disclosed in further detail below, the microfluidic chips of the present invention are capable of capturing and concentrating genetic material for the analysis and identification of biological organisms, such as the so-called “threat genes” from biological weapons. The microarrays are capable of being in fluid communication with the functionalized porous polymer monolith to provide microfluidic chips that are capable of capturing thousands of expressed genes, such as mRNA. These features enable a reduction in sample preparation time, a reduction in required sample volume, an increase in sensitivity, and decreased sample degradation. All of these characteristics are important for the effective use and operation of portable bioweapons detectors by both military and civilian personnel. Further uses of the described technology include the detection of infectious and hazardous biological agents in a clinical setting. The described invention has the capability of rapidly detecting thousands of infectious agents in complex matrices such as blood, food products, and complex environmental samples.

Within additional aspects there are provided microfluidic chips that include a plurality of vias; a functionalized porous polymer monolith capable of being in fluid communication with a via; a microarray capable of being in fluid communication with the functionalized porous polymer monolith; one or more mobile monolith valves capable of controlling fluid flow in the microfluidic chip; and an observation port through which at least one target disposed within the microarray is capable of being detected. The mobile monolith valves assist the fluidic operation of the microfluidic chips, such as controlling the capture and concentration of targets in the functionalized porous polymer monoliths.

### BRIEF DESCRIPTION OF THE DRAWINGS

The invention may be understood by reference to the following description taken in conjunction with the accompanying drawings, in which:

FIG. 1 shows a schematic of one embodiment of the present invention of a microfluidic chip having a two-dimensional microarray, two porous polymer reservoirs, and microchannels connected to eight vias.

FIG. 2 shows a schematic of one embodiment of the microfluidic chip of the present invention having a two-dimensional microarray, two porous polymer reservoirs, and microchannels connected to eight vias. This schematic further depicts pressure-equalizing manifolds in fluid communication between the vias and the porous polymer reservoirs.

FIG. 3 shows a schematic of the detail of the layout of the vias and observation port of the microfluidic chip of the present invention.

FIG. 4 shows a schematic of the detail of the layout of a via, a microchannel, a portion of a pressure-equalizing manifold, and a portion of a microarray of one embodiment of the microfluidic chip of the present invention.

FIG. 5 shows a schematic of the detail of the layout of a portion of a pressure-equalizing manifold, and a portion of a microarray of one embodiment of the microfluidic chip of the present invention.

FIG. 6 shows a schematic of the detail of the layout of a portion of a pressure-equalizing manifold, and a portion of a functionalized porous polymer monolith reservoir of one embodiment of the microfluidic chip of the present invention.

FIG. 7 shows a schematic of the detail of the layout of a via and a microchannel of one embodiment of the microfluidic chip of the present invention.

FIG. 8 depicts the layout of multiple microfluidic chips prepared on a single substrate.

FIG. 9 is a perspective view of a microfluidic chip having cover and base substrates. Depicted are microchannels and regions reserved for the gene spotting microarray and functionalized porous polymer monoliths in the bonding plane of the microfluidic chip. Access to the region reserved for the two-dimensional microarray is through an observation port in the cover substrate.

FIG. 10 is a perspective view of a microfluidic chip having cover and base substrates. Depicted are microchannels and regions region reserved for the functionalized porous polymer monoliths in the bonding plane of the microfluidic chip. Microchannels are routed vertically through vias to open channels on the top of the cover substrate. The two-dimensional microarray region for the gene spotting area is a channel on top of the cover substrate.

FIG. 11 is a perspective view of a microfluidic chip having cover and base substrates. Depicted are microchannels and regions reserved for the functionalized porous polymer monoliths in the bonding plane of the microfluidic chip. Microchannels are routed vertically through vias to open channels on the top of the cover substrate. The microarray region for the gene spotting area is a one-dimensional serpentine channel on top of the cover substrate.

FIG. 12 shows a schematic of one embodiment of the present invention of a microfluidic chip having a one-dimensional serpentine microarray laid out in a square formation, two porous polymer reservoirs, and microchannels connected to eight vias.

FIG. 13 shows a schematic of one embodiment of the present invention of a microfluidic chip having a one-dimensional circular serpentine microarray, two porous polymer reservoirs, and microchannels connected to eight vias.

FIG. 14A depicts a porous polymer monolith, pre-functionalized.

FIG. 14B depicts a nonfunctionalized porous polymer monolith imaged using a fluorescent microscope at 488 nm.

FIG. 14C depicts binding of a fluorescent-tagged amine molecules, oligo(dT), to a functionalized porous polymer monolith functionalized imaged using a fluorescent microscope at 488 nm.

### DETAILED DESCRIPTION OF ILLUSTRATIVE EMBODIMENTS

Terms

The term “microchannel” as used herein is intended to be synonymous with the term “microfluidic channel”. Microchannels may be filled with or may contain internal structures comprising, for example, valves, filters, or equivalent components and materials. A microchannel has a dimensional feature that is at least about 1 micron but is less than about 500 microns in size. During operation, a microchannel may contain a fluid passing therethrough.

The term “fluid” as used herein refers to matter that flows under the influence of a pressure gradient. Examples of fluids include gases, liquids, suspensions, emulsions, aerosols and mixtures thereof

The term “microfluidic” as used herein describes structures or devices through which a fluid is capable of being passed or directed, wherein one or more of the dimensions is less than about 500 microns.

The term “microfluidic chip” as used herein refers to at least one substrate having microfluidic structures contained therein or thereon.

The term “via” as used herein refers to a fluidic passage between substrates of a microfluidic chip or between a substrate of a microfluidic chip and other fluidic structures exterior to the substrate which are in fluidic communication.

The term “sample inlet via” as used herein refers to a via through which analyte compounds enter the microfluidic chip.

The term “capable of being in fluid communication” as used herein refers to the ability of a fluid to move from one location to another.

The term “microarray” as used herein refers to a collection of probes synthesized, attached or deposited on a substrate.

The term “probe” as used herein refers to a molecule synthesized, attached or deposited on a microarray that can be recognized by a target.

The term “target” as used herein refers to a molecule to which a probe is designed to specifically bond with.

The term “observation port” as used herein refers to a region on a microfluidic chip that permits detection of targets within a microarray.

The term “mobile monolith valve” as used herein refers to the devices that control and regulate fluid flow in microfluidic systems by means of a mobile, monolithic polymer element, as disclosed in U.S. Patent Application Pub. No. U.S. 2002/0194909, “Mobile Monolithic Polymer Elements for Flow Control in Microfluidic Devices”, the disclosure of which is incorporated by reference in its entirety.

The term “porous polymer monolith” as used herein refers to the highly crosslinked monolithic porous polymer materials described in U.S. Pat. No. 6,472,443 to Shepodd, the disclosure of which is incorporated by reference in its entirety.

The term “functionalized porous polymer monolith” refers to porous polymer monoliths having chemical functions on the surfaces of the pores that are capable of contacting and bonding to analytes passing through the pores.

The term “nucleic acid” refers to a deoxyribonucleotide or ribonucleotide polymer or oligomer in either single-or double-stranded form.

The terms “RNA” and “DNA” as used herein refer to ribonucleic acid and deoxyribonucleic acid, respectfully.

The term “mRNA” refers to “messenger RNA”, i.e., transcripts of a gene. Transcripts are RNA including, for example, mature messenger RNA ready for translation and products of various stages of transcript processing. Transcript processing may include splicing and degradation.

The term “target nucleic acid” refers to a nucleic acid to which a probe is designed to specifically hybridize. The target nucleic acid has a sequence that is complementary to a nucleic acid sequence of a corresponding probe directed to the target.

The term “oligonucleotide” refers to a single-stranded nucleic acid ranging in length from 2 to about 500 nucleotide bases.

The term “plurality” as used herein refers to two or more. Unless otherwise indicated, an attribution to one in the plurality does not necessarily apply to the other(s) in the plurality.

All ranges disclosed herein are inclusive and combinable.

The microfluidic chips of the present invention typically include a plurality of vias, a functionalized porous polymer monolith capable of being in fluid communication with at least one of the vias, a microarray capable of being in fluid communication with the functionalized porous polymer monolith, and an observation port through which at least one target disposed within the microarray is capable of being detected. The microfluidic chips of the present invention are typically constructed using one or more substrates. Substrates are typically made from a transparent material to aid observation, however non-transparent materials can be used. Suitable transparent substrate materials include glass, silicon, silicon nitride, quartz, and preferably fused silica. Other substrate materials that can be used include various materials, such as glass, polymeric, ceramic, metallic, and composite materials, as well as combinations thereof. A variety of microstructural fluidic elements can be prepared on substrates using standard wet-etching photolithography procedures. A plurality of vias in the microfluidic chips are typically provided to transport fluids into, out of, and onto the various microfluidic structures within the microfluidic chips, or any combination thereof. Vias can be prepared using standard wet etching procedures, but are typically provided by the use of a diamond tipped drill, such as a microdrill. In various embodiments of the present invention, microfluidic chips include two substrates (e.g., a cover substrate and a base substrate) that are bonded together. The bonding of the substrates, which may be adhesive bonding, cohesive bonding, or both, provides regions for containing microfluidic structures in the base substrate and a plurality of vias in a cover substrate. When bonded together, the spatial arrangement of the vias in the cover substrate are typically designed to be in fluid communication with the regions containing the microfluidic structures.

The microfluidic chips of the present invention contain a region for providing at least one microarray. The microarrays are provided in regions of the microfluidic chips where the targets can be detected. These regions for target detection can be provided within the microfluidic chip, on the microfluidic chip, or both. The regions for target detection suitably require at least several square centimeters in area of the microfluidic chips for the detection of targets, but they can be smaller or larger depending on the number, size and type of probes used. Typically, the microarrays include at least one probe which is capable of binding at least one target. Targets are typically a compound or molecule, which when detected, provides information about the origin or nature of a biological sample. Suitable targets typically include a nucleic acid, a protein, an antigen, an antibody, or any combination thereof. Nucleic acid targets typically include RNA, DNA, LNA, PNA, HNA, or any combination thereof, which are capable of hybridizing with a nucleic acid on the probe. More preferably, the probes are capable of hybridizing with DNA target molecules, preferably cDNA, as described further below. Accordingly, suitable probes for hybridizing with cDNA will typically include nucleic acids such as oligonucleotides. Although it is preferred that oligonucleotide probes are provided as single stranded nucleic acids, double stranded nucleic acids as well as combinations of single and double stranded nucleic acids can also be used.

Suitable microarrays typically include a plurality of probes that are capable of binding a plurality of targets. Typically, though there is no lower size limit, the microarray will include at least about 100-1,000 probes, more typically at least about 5,000 probes, and even more typically at least about 10,000 probes. Greater numbers of probes can be placed on microfluidic chips, especially as the size of separation of the probes decreases and the size of the microarray increases. Accordingly, there is theoretically no upper size limit of the chips. Typically, however, microarrays will have up to about 50,000 probes. Typically the plurality of probes will include at least one probe different than the other probes. Different probes permit the detection of different targets. In this regard, different probes are typically provided that are capable of binding different targets. Although several of the probes may bind two or more different targets, it is more typical that each of the probes is individually capable of binding a different target.

In one embodiment of the present invention, a DNA based detection system, such as one capable of being fashioned on a 1×3 inch glass slide is provided as a suitable microarray for the microfluidic chip. DNA sequences can be deposited using a robotic spotter or photolithography in the microarray region of the microfluidic chip to provide between about 1000 to about 300,000 gene spots per slide for a robotic spotter and up to about 1,000,000 gene spots using photolithography. Higher number of gene spots are envisioned as the size of the microarray increases, as the spot size decreases, or both. In use, the microarray is designed to detect the presence of specific genes, such as the expressed gene's (MRNA) of a variety of biological samples. Suitable biological samples include animal blood or tissues, plant, tissues, bacteria, mold, spores, and viruses.

In certain embodiments of the present invention, at least one of the probes for the microarray is disposed as at least one spot on the surface of a base substrate of the microfluidic chip. Preparing probe spots in the regions containing the microarrays can be carried out by means of an automated robotic spotting device, such as is described by Schena, M., et al., Quantitative monitoring of gene expression patterns with a complementary DNA microarray, Science, 1995, 270 (5235), 467-70. These devices can be constructed for spot deposition for custom architectures such as the described microfluidic microarray. For use on conventional glass slide microarray robotic spotting instruments are commercially available from Amersham Biosciences (San Francisco, Calif.), Packard Biosciences (Palo Alto, Calif.), Gene Machine, Inc. (San Carlos, Calif.) and TeleChem International (Sunnyvale, Calif.). The probes can be contained in the microarrays as a spot that is typically at least about 10 microns wide, more typically at least about 20 microns wide, even more typically at least about 40 microns wide, and further typically at least about 60 microns wide. Although there is no upper spot size limit, in various embodiments of the microfluidic chips of the present invention the microarrays will have spots at most about 250 microns wide. Suitable spots may be of any shape, including regular shapes such as a triangle, square, hexagon, or a circle, and irregular shapes too. The spots of the probes are typically circular in shape.

For embodiments containing microarrays having a plurality of probes that are disposed as a plurality of spots on the surface of a base substrate, the plurality of spots are typically separated from one another so that no two spots are on average closer than a particular distance, this distance typically being at least about 10 microns, more typically being at least about 20 microns, even more typically being at least about 50 microns, and further typically being at least about 100 microns. As used herein, the term “no two spots are on average closer than a particular distance” is intended to mean that a portion of the spots may be closer than the indicated distance as long as the average of all the pair-wise nearest-neighbor spot distances is not smaller than the indicated distance. Although various microfluidic chips can have spots that are separated by their nearest neighbors of average distances greater than about 500 microns, typically the average distance between two neighboring spots will be less than about 500 microns.

The probes can be arranged in any fashion, either without any apparent order (i.e., disordered), but is typically provided as an ordered microarray. In an ordered microarray, the spacing between nearest neighbor probes is regular and low in variability. In contrast, the spacing between nearest neighbor probes in a disordered array is typically irregular and high in variability. In the embodiments in which the microarray contains an ordered arrangement of probes, a disordered arrangement of probes, or both types of arrangements, the type and location of the probes will be typically known. The probes in the microarray are typically arranged in one, two or three dimensions. A one dimensional microarray typically includes a plurality of probes that are linearly arranged, as in a straight line. The microarray probes may also be linearly arranged in two dimensions, such as in a serpentine path.

In several embodiments of the present invention, the microarrays of the microfluidic chips have a plurality of probes that are disposed as a plurality of spots on the surface of a base substrate. In these embodiments, the plurality of spots are typically disposed within at least one microchannel, which forms a linear region for the microarray that is suitable for receiving probe spots. Linear microarrtays can suitably be prepared in a microchannel that is from about 10 microns to about 500 microns wide and from about 1,000 microns to about 1,000,000 microns long. Longer linear microarrays are envisioned for substrates larger than about 25 millimeters by 32 millimeters. Although the microchannel may be disposed in a straight line that does not vary appreciably in direction along the microfluidic chip, a microarray can include at least one microchannel that varies in direction along the surface of the substrate. Accordingly, the at least one microchannel can be disposed as a spiral path, a serpentine path, a curved path, or as a straight path. Two or more microchannels can also be suitably linked to provide one or more microarrays. In these embodiments a microchannel will be in fluid communication with at least one other microchannel having a spiral path, a serpentine path, a curved path, or a straight path, or any combination thereof. For microarrays having a serpentine design, the serpentine design can include a circular serpentine path, a rectangular serpentine path, or any combination thereof. Serpentine path microchannels are particularly preferred as microarrays as they help contain analyte flow in a region in proximity to the probes. Increasing the path length of linear microarrays permits an increase in the number of probes contained therein. Accordingly, a serpentine path will desirably be disposed in a fashion the minimizes the amount of needed area of the microfluidic chip. Thus, a suitable serpentine path will include a first section that is disposed adjacent to a second section of the serpentine path, the first and second sections being separated by a wall of non-zero thickness. By “non-zero wall thickness” is meant that one section of a serpentine path is physically separated (e.g., by a wall of substrate material) from a different section of a serpentine path, the different section being down stream or up stream from the same'serpentine path, or the different section being on a different serpentine path. In several embodiments, the thickness of the wall is typically in the range of from about 10 microns to about 1,000 microns.

In another embodiment of the present invention there is provided a microfluidic chip wherein the plurality of probes are planarly arranged in two dimensions. As used herein, the term “planarly arranged” is meant to refer to the probes being arranged two dimensionally, such as substantially on a common plane or surface. Accordingly, in several embodiments of the present invention, the microarrays can have a plurality of probes that are disposed as a plurality of spots on the surface of a base substrate. Here, the plurality of spots are suitably arranged in rows and columns numbering from about 10 to about 1,000. Typically, a microwell region is provided in a microfluidic chip to contain a planar microarray in which the spots are disposed.

In several embodiments wherein the microfluidic chip is prepared from the bonding of base and cover substrates, the plurality of probes are typically disposed as a plurality of spots on the surface of the base substrate. In these embodiments the cover plate can comprise an open portion above the microarray region for direct spotting onto the microarray region of the base substrate surface. The plurality of spots can be disposed on the base substrate in the microarray region in a fashion wherein the mean distance between the plurality of spots is in the range of from about 10 to 500 microns. In these embodiments, placement of the probes (i.e., spots) on the base substrate is carried out using probes that are capable of binding the at least one target. The number of molecules sufficient to form a spot of a particular size is typically determined as the amount necessary to form at least about one monolayer of the probe molecules for that spot. Preferably, the probes comprise nucleic acids capable of hybridizing with at least one target corresponding to a biological sample. More preferably, the probes are covalently bonded to the substrate, either directly, or by way of a linker molecule, or other substrate coating that covalently bonds the probe and the substrate.

It is also envisioned that the microfluidic chip may contain a plurality of probes that are spatially arranged in three dimensions, such as those disclosed by Cheek et al., Analytical Chemistry, Vol. 73, No. 24, 5777-5783 (2001). Cheek et al. describe chemiluminescence (“CL”) detection for hybridization assays on a “Flow-Thru” chip, which is a three-dimensional microchannel biochip. Such a “Flow-Thru” chip can be fashioned on the microfluidic chips of the present invention.

A functionalized porous polymer monolith is typically provided to carry out capture and concentration of biological sample compounds. These sample compounds can be subsequently treated for further identification and analysis using the microarray. The synthesis and preparation of non-functionalized porous polymer monoliths is provided in U.S. Pat. No. 6,472,443 to Shepodd (“the Shepodd patent”), the disclosure of which is incorporated by reference herein. Functionalization of the porous polymer monoliths is preferably carried out by post-functionalizing the porous polymer monolith, as described further below. Alternatively, functionalization can be carried out by including a polymerizable functionalized monomer in a reaction mixture for preparing porous polymer monoliths. The functionalized monomer is selected to contain a functional group that directly binds particular target biological compounds, or to directly bind probe compounds capable of selectively binding to particular target biological compounds. Suitable probe compounds include an amine-containing ligand, or any nucleophilic residue that is located on one terminus of the probe to be covalently attached to the array surface which has been functionalized with and electrophilic species such as epoxide or aldehyde bond. In this arrangement a nucleic acid, a protein, an antibody, an antigen or cell receptor ligand or cell receptor, or any combination thereof can be covalently attached to the array surface. Preferably the probe compounds directly bindable to the functional groups include oligonucleotides, proteins, whole organisms (bacteria, viruses), or individual cells that are capable of binding these specific targets. An example of this arrangement in the covalent amine-aldehyde linkage of a complementary oligonucleotide probe to the array surface. Detection of the probe complement is facilitated by the hybridization (or the formation of a stable double stranded DNA complex) on the array surface using a target that has been labeled with a fluorescent molecule.

Typically the functional group of the functionalized porous polymer monolith is capable of binding a nucleic acid. A particularly preferred nucleic acid that is capable of binding expressed genes in a biological sample is oligo-T (i.e., for hybridization of poly-A segments of mRNA). Accordingly, amine-containing oligo-T can by bound to porous polymer monoliths through a monomer that is capable of copolymerizing with the porous polymer monolith and which also includes a functional group capable of forming a covalent bond with oligo-T. Examples of suitable functional groups capable of binding oligo-T include glycidyl, or aldehyde chemistries. Accordingly, suitable monomers include, ethylene glycol dimethacrylate, 2-hydroxyl ethyl methacrylate, tetrahydroxyl furan methacrylate, lauryl acrylate, morpholine acrylate, 2-hydroxy ethyl acrylate, and preferably glycidyl methacrylate (“GMA”). Typically, the functionalized porous polymer monolith includes pores having a surface, the pores permitting fluid communication through the functionalized porous polymer monolith. The functionalized porous polymer monolith also typically includes a highly crosslinked polymer. A variety of crosslinked polymers can be prepared by the methods disclosed in the Shepodd patent, but typically the highly crosslinked polymer includes units derived from at least one mono-ethylenically unsaturated monomer, at least one multi-ethylenically unsaturated monomer, or a combination thereof. Examples of suitable mono-ethylenically unsaturated monomer include any of the mono-ethylenically unsaturated, functionalized or unfunctionalized, acrylic or methacrylic monomers known in the polymer art, such as 2-hydroxyethyl acrylate, 2-hydroxyethyl methacrylate, glycidyl methacrylate and 9-anthracenylmethyl methacrylate. Other suitable mono-ethylenically unsaturated monomers include allylglycidyl ether, 2-vinyl oxirane, and polybutadiene-maleic anhydride. Examples of functional groups include inter alia alcohol (e.g., hydroxyethylmethacrylate) and glycidyl (e.g., glycidyl methacrylate). Examples of multi-ethylenically unsaturated monomer include ethylene glycol dimethacrylate (“EGDMA”), polyethyleneglycol dimethacrylate, tetraethyleneglycol dimethacrylate, triethyleneglycol dimethacrylate, ethylene dimethacrylate, 1,3-butanediol dimethacrylate, 1,4-butanediol dimethacrylate, 1,6-hexanediol diacrylate, tripropyleneglycol diacrylate, trimethylolpropane triacrylate (“TMPTA”), trimethylolpropane trimethylacrylate (“TMPTMA”). Typically, the functionalized porous polymer monoliths are prepared by using a suitable UV photo initiator, such as Irgacure™ 1800.

While the microfluidic chips of the present invention can comprise any type of functionalized porous polymer monolith as described herein, it is desirable that this monolith includes pores that are smaller than about 20 microns, typically between 0.1 and 10 microns. Larger pore size is typically accompanied by a reduction in contact area, and therefore a reduction in binding capacity. Larger pore sizes lead desirably to lower pressure differential. Also, it is desirable that the functionalized porous polymer monolith comprises a void fraction of less than about 50 percent based on volume of the functionalized porous polymer monolith. Decreasing the void fraction is typically accompanied by a high pressure differential and an increase in binding capacity. Also, suitable functionalized porous polymer monoliths are capable of providing a pressure drop in the range of from 100 to 3000 psi to an aqueous fluid at 25° C. that is communicated therethrough. The functionalized porous polymer monolith is typically covalently bonded to the microfluidic chip substrate. Without being bound by a theory of operation, it is believed that this covalent bonding prevents portions of the monolith from migrating through the microfluidic chip. Functionalized porous polymer monoliths that are fixed within the reservoirs of the microfluidic chips typically do not require the use of frits for the purposes of containing the monoliths.

In several embodiments of the present invention, microfluidic chips are provided, wherein at least one of the microarray and the functionalized porous polymer monolith are disposed between a base substrate and a cover substrate. Preferably both of the functionalized porous polymer monolith and the microarray are disposed between a base substrate and a cover substrate. In several of these embodiments, the plurality of vias are disposed within the base substrate, the cover substrate, or any combination thereof, the vias being in fluid communication with the functionalized porous polymer monolith, the microarray, or both. In addition, at least a portion of the vias are capable of being in fluid communication with fluidic devices external to the microfluidic chip. The vias enable the microfluidic chip to be integrated with a suitable analytic device capable of delivering analytes, sample compounds, carrier fluids, and any combination thereof to and away from the microfluidic chip. Such analytical devices incorporating microfluidic chips are disclosed in U.S. patent application Ser. No. 10/633,871, filed Aug. 4, 2003, “Portable Apparatus for Separating Sample and Detecting Target Analytes”, Attorney Docket No. SD-8412.1, the disclosure of which is incorporated by reference in its entirety.

In another embodiment, a microarray is disposed on a top surface of the cover substrate. In this embodiment the cover substrate comprises a region above the microarray to provide the observation port.

In another embodiment is provided a microfluidic chip, wherein at least one of the vias is not in fluid communication with the functionalized porous polymer monolith. For example, fluid delivery to the microarray can be carried out by use of an external fluid reservoir that transports fluid through an inlet via into the microarray, and then from the microarray through an exit via to a waste reservoir. In this embodiment the inlet and exit vias fluidically isolate the microarray from the functionalized porous polymer monolith. Accordingly, this embodiment provides that the functionalized porous polymer monolith is not in fluid communication with the microarray.

In another embodiment is provided a microfluidic chip, wherein the base substrate and the cover substrate are at least partially bonded together at a bonding surface. For fused silica substrates, the bonding surface is typically prepared by aligning and pressing the base and cover substrates. Thermal bonding is then accomplished by heating the aligned wafers at 875° C. for one hour, then 1100° C. for five hours, then allowing the wafer to cool to room temperature over a period of twelve hours. The resulting interface between the base and cover substrates is referred to as the bonding surface. The term “at least partially bonded together at a bonding surface” as used herein refers to the absence of bonding between the two substrates arising from the presence of fluidic structures on one or both surfaces, or due to the presence of vias and windows in one or both of the substrates.

Typically the base substrate includes at least one microfluidic structure disposed at the bonding surface. Examples of microflulidic structures include a microchannel, a microwell, a reservoir, a microelectrode, a microjunction, a microsplitter, a microfilter, a microreactor, a microvalve, a microsensor, a microinjector, a micromixer, a micropump, a microseparator, a micromanifold, or any combination thereof. A reservoir is typically formed between the base and cover substrates by etching a region of from about several square millimeters to about several square centimeters into a base substrate. The etched reservoir region is covered and thermally bonded to a cover substrate. Other microfluidic structures that can be formed this way include microchannels, microwells, or any combination thereof. As used herein the terms “microwell” and “microchamber” are synonymous. Microfluidic structures that span a large area in which flexing of the cover substrate may occur will typically further include microposts bonded between the base substrate and the cover substrate. Microposts are capable of reducing the deformation of the cover substrate disposed above the microfluidic structure. Microposts are also capable of providing mixing to fluids flowing around the microposts, or through the microfluidic structure, or both. Microposts can also be used in various embodiments of the present invention for the purposes of controlling mixing and pressure in microfluidic structures.

The microfluidic structures that are disposed at the bonding surface are characterized as including a region of finite dimensions in the vicinity of the bonding surface. These dimensions are characterized as dimensions perpendicular to the bonding surface and dimensions parallel to the bonding surface. The perpendicular dimension is typically up to about 1,000 microns, more typically in the range of from about 1 to about 500 microns, even more typically in the range of from about 5 to 250 microns, and even further typically in the range of from about 10 to 100 microns. The parallel dimension is typically up to about 100,000 microns, more typically in the range of from about 10 to about 50,000 microns, even more typically in the range of from about 50 to about 25,000 microns, and even further typically in the range of from about 100 to 10,000 microns. Various embodiments of the present invention will typically have a base substrate that includes a plurality of microfluidic structures in the bonding surface, the preferred microfluidic structures including a micromanifold, microarray, microchannel, a microwell, a reservoir, a microelectrode, or any combination thereof.

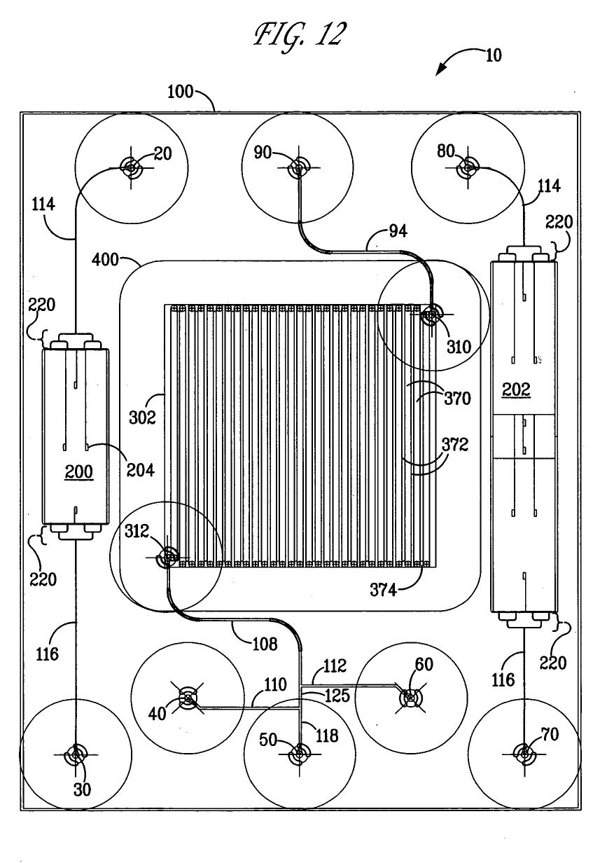

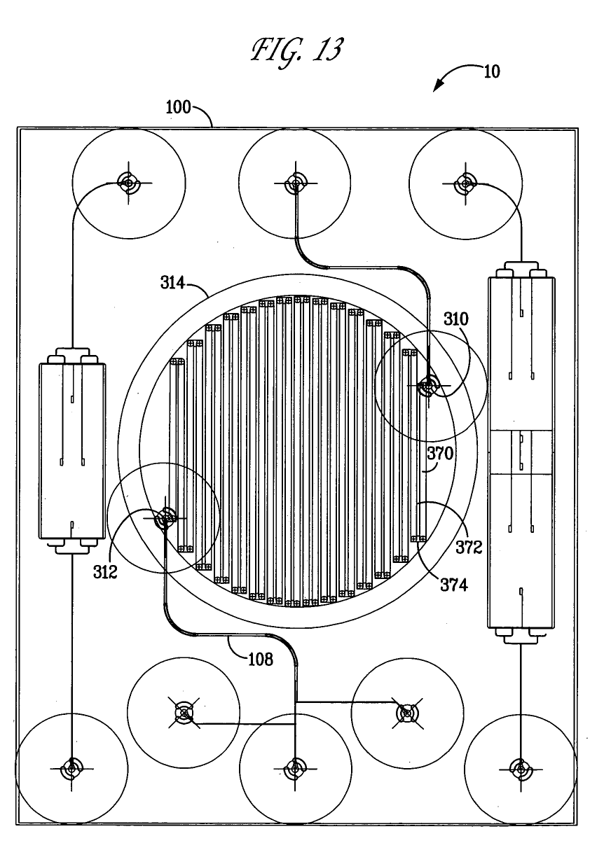

FIG. 12 and FIG. 13 depict embodiments of the present invention in which the base and cover wafers are prepared to provide structures that reside between the bonded wafers, and which reside on the device surface. In these embodiments, the probes are deposited on the device surface after the device is bonded at the annealing temperature. The sample preparation channels are wet etched on the bottom wafer and a single open channel is wet etched on the top wafer. The design architecture of this embodiment provides the ability to form pressure tight sample preparation channels in the interior of the microfluidic device which the sample preparation monolith can be polymerized, functionalized and used for trapping and labeling of target analytes. This embodiment also provides a serpentine or open channel area which can be fabricated on the surface of the microfluidic device. In this arrangement, probes can be deposited on the surface of the microfluidic using a robotic spotter after device fabrication is complete. The serpentine or open channel area can then be sealed with an appropriate coverslip or detection platform to observe the hybridization of the complementary target sequences or analytes of the probe sequence deposited on the array surface.

In one embodiment of the present invention, the microfluidic chip includes at least one microfluidic structure (e.g., a microwell, a reservoir or a region for containing a microarray), which further includes a micromanifold that is capable of equalizing the pressure distribution, the flow distribution, or both within the microfluidic structure. A micromanifold typically includes a plurality of branched microchannels that are in fluid communication with each other. A micromanifold typically includes a main trunk microchannel that is branched to a first series of two or more microchannels. Subsequent branching of each microchannel of the first series can be branched into a second series of two or more microchannels, and so on. The number of branches and series typically will depend on the ratio of the cross-sectional flow field area of the microfluidic structure to that of the trunk microchannel. The larger this ratio the greater the number of branches of the micromanifold that are typically required to equalize the pressure and flow distributions. Suitable micromanifolds typically have from one to about ten, more typically from one to about five, and even more typically from about one to four series of branches. Each series typically includes from two to about five branches, and more typically from about two to four branches. Micromanifolds having a combination of series and branches can also be used.

In one embodiment of the present invention the microfluidic chip includes a microfluidic injector in fluid communication with a microfluidic structure, the microfluidic injector being capable of providing a fluid plug into the microarray. A microfluidic injector is typically provided using a fluid inlet via, an injector microchannel in fluid communication with the fluid inlet via, the injector microchannel truncating at an inlet microchannel which leads towards the microarray. In alternate embodiments the inlet microchannel leads directly into the microarray region or it first leads into a micromanifold that subsequently branches into the microarray region.

In another embodiment of the present invention there is provided a microfluidic chip that further includes a derivatization reservoir which contains the functionalized porous. polymer monolith. The derivatization reservoir is typically 1-4 mm×6-10 mm in dimension, and the reservoir for the larger monolith is typically 1-4 mm×10-20 mm in dimension. In this embodiment, the derivatization reservoir includes the post-functionalized porous polymer monolith which has the ability trap and concentrate the target analyte such as a nucleic acid or protein. After trapping is complete, a second solution containing suitable reagents for fluorescent labeling of the target analyte is introduced. Several strategies can be used to complete the labeling of the trapped target analyte. In one arrangement, a complementary cDNA can be synthesized using the trapped target analyte as a template. In this arrangement free nucleic acid bases containing a fluorescent molecule and an enzyme such as DNA polymerase for making a complementary cDNA copy of DNA, or reverse transcriptase for making a complementary cDNA copy of RNA, or both, can be used to produce a fluorescent cDNA sequence. The resulting fluorescent CDNA sequence is capable of specifically interrogating the presence of a target analyte. In a another arrangement a chemical moiety (as described in Houtoff, H., et al., Platinum-containing compounds, methods for their preparation an applications thereof, 1999, Kreatech Diagnostics: Netherlands), is introduced to the chamber that can electophilically attack the trapped target analyte which accomplishes the fluorescent labeling of target analyte without removing the trapping target analyte. In this arrangement, the target analytes are directly labeled using a chlorinated platinum compound that is chemically modified to contain a fluorescent dye molecule. This platinum molecule specifically reacts at the N7 position of the guanine residue and to a lesser extent to the N7 position on an adenine nucleic acid base to form a covalent bond with the trapped target analyte. These labeled target nucleotides can then be eluted using either low salt containing solutions, or alternatively, by heating the derivatization channel to temperatures above 80° C. Suitable molecular labels, such as fluorescent tags, can be purchased from commercial vendors, such as Amersham Biosciences or Molecular probes. In additional embodiments, the derivatization reservoir containing the polymer monolith may include a variety of nucleotide sequences that can target specific groups of gene families. Many nucleotide sequences can be provided to selectively concentrate a variety of biomolecules, such as: target sequences from an extremely complex sample; proteins that recognize specific gene or other protein target; and ligands that can selectively bind cell surface receptors or any combination thereof.

In another embodiment of the present invention there is provided a microfluidic chip that further includes one or more mobile monolith valves capable of controlling fluid flow within, into, out of, or onto the microfluidic chip, or any combination thereof. The preparation and use of mobile monolith valves in microfludic chips is described fully in U.S. Patent Application Publication No. 2002/0194909, “Mobile Monolithic Polymer Elements for Flow Control in Microfluidic Devices”, to Shepodd, the entire disclosure of which is incorporated by reference thereto. Typically, a microfluid control device, or microvalve, is made that includes generally a cast-in-place, mobile monolithic polymer element, disposed within a microchannel, and driven by a displacing force that can be fluid (either liquid or gas) pressure or an electric field against a sealing surface, or retaining means that can include a constriction or a stop in the microchannel, to provide for control of fluid flow. As a means for controlling fluid flow, such microvalves possess the additional advantage that they can be used to effect pressure and electric field driven flows, eliminate or enhance diffusive or convective mixing, inject fixed quantities of fluid, and selectively divert flow from one channel to various other channels. They can also be used to isolate electric fields, and, as a consequence, locally isolate electroosmotic or electrophoretic flows.

The mobile monolith polymer elements are not restricted to any particular shape or geometry except by the configuration of microchannel in which it functions and the requirement that they provide an effective seal against fluid flow for valving applications. By providing a method for producing a monolithic polymer element that does not bond to surrounding structures, these polymer elements are free to move within the confines of a microchannel and can be translated within the microchannel by applying a displacing force, such as fluid pressure or an electric field to the polymer element. Additional fluid flow control, regulation, and distribution devices that can be included in various embodiments of the microfluidic chips also include, but not limited to, nano- and pico-liter pipettes and syringes needle valves, diverter valves, water wheel flowmeters, and flow rectifiers.

The mobile polymer monolith microvalves are typically fabricated by photoinitiating phase-separation polymerization in specified regions of a three-D microstructure, typically glass, silicon, or plastic. Functionality is achieved by controlling monolith shape and by designing the polymer monoliths to move within microfluidic channels. In-situ fabrication of the polymer monoliths typically assures that their shape will conform to the microchannel geometry.

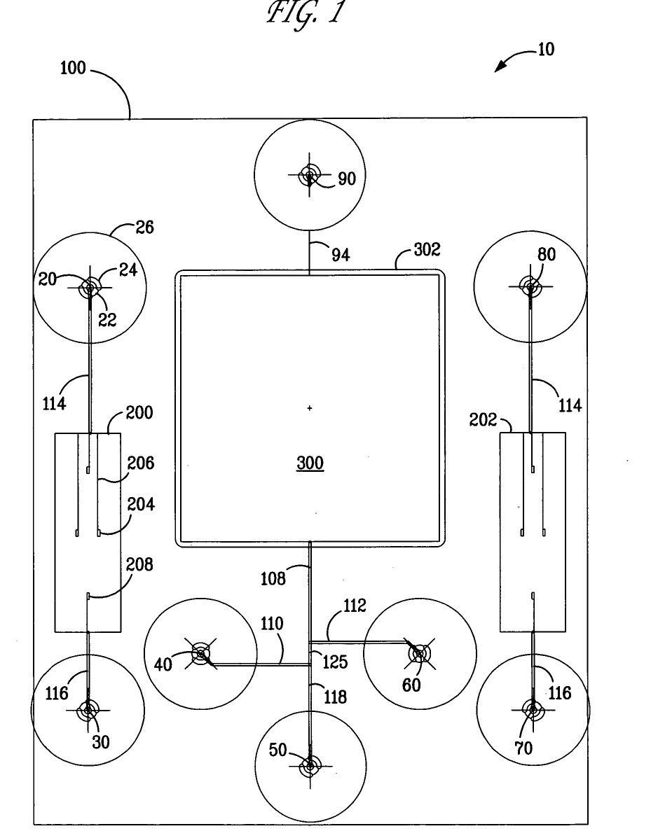

FIG. 1 depicts one embodiment of a microfluidic chip according to the present invention. Microfluidic chip 10 includes a base substrate 100 upon which a number of microstructural features are provided. A plurality of vias 20, 30, 40, 50, 60, 70, 80 and 90 are provided to transport fluids into and out of reservoirs 200 and 202 through microchannels 114 and 116. At least one of the reservoirs 200 and 202, contains a functionalized porous polymer monolith. Surrounding each of the vias, as depicted for via 20, is a circular region 22 for placement of an O-ring 24 for connecting fluidic structures (e.g., reagent reservoirs, sample reservoirs, pumps, and tubing) to the microfluidic chip. Fittings for connecting external fluidic structures contact the circular regions around each of the vias, such as demarcated by circle 26. Vias 20 and 30 are in fluidic communication with reservoir 200 through microchannels 114 and 116, respectively. Reservoir 200 further has support posts 204 and 208 to support a cover substrate (not shown), which cover wafer to prevent the reservoir from collapsing under physical pressure. Via 50 is fluidically connected to microarray region 300 that is contained with the region situated with microarray wall 302 through microchannels 108 and 118. As shown, reservoirs 200 and 202 and microarray 300 are not in fluidic communication on the microfluidic chip, but are capable of being in fluidic communication through fluidic structures external to the microfluidic chip. During use of microfluidic chip 10, via 50 is typically fluidically connected to an external buffer reservoir that is transported into the microfluidic chip through via 50. Fluids are typically transported into the microfluidic chip under the influence of hydrostatic, electroosmotic, or electrophoretic forces, or any combination thereof. Other fluids, such as sample fluids containing targets, can be load as a fluid plug into the microchannel injector region 125 situated between microchannels 108 and 118, which are situated between the junctions with microchannels 110 and 112. Fluids exit the microarray 300 through microchannel 94 and out of the microfluidic chip through waste via 90. The control of fluids into microfluidic chips is described in further detail in U.S. patent application Ser. No. 10/633,871, filed Aug. 4, 2003, “Portable Apparatus for Separating Sample and Detecting Target Analytes”, Attorney Docket No. SD-8412.1, the disclosure of which is incorporated by reference in its entirety.

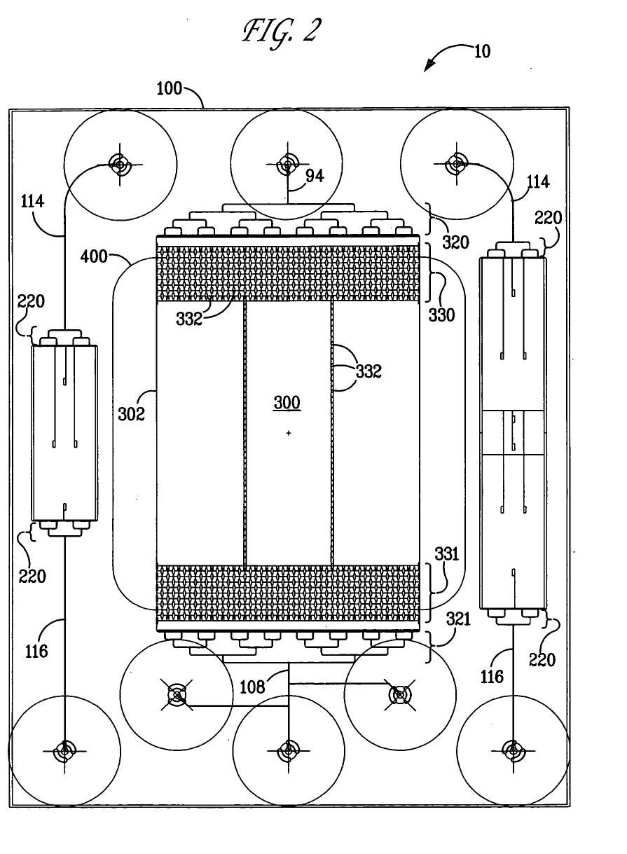

FIG. 2 shows another embodiment of the microfluidic chip of the present invention. In this embodiment, microfluidic chip 10 includes structures similar to those depicted in FIG. 1, and further includes micromanifolds 220 for two reservoirs, reservoir 200 being a smaller reservoir of approximate dimensions 3 millimeters by 8 millimeters and reservoir 202 being a larger reservoir of approximate dimensions 3 millimeters by 16 millimeters, micromanifolds 320 and 321 for the microarray region 300, and micropost arrays 331 situated within the microarray region 300. Although not drawn to scale, the substrate 100 in this embodiment is approximately 25 millimeters wide by 32 millimeters long by about 1 millimeter thick. The eight vias are approximately 300 microns wide. The microchannels have a depth that is between about 10 and 40 microns. During operation, fluids are transported through microchannel 108 into micromanifold 321, which contains four series of branches, each series having two branches, to provide a total of 2 branches raised to the power of 4 series, or sixteen total branches that enter microarray 300. Fluid transported from microchannel 108 is divided in the micromanifold to more evenly flow into microarray region 300. The microarray region 300 further includes a micropost region 331 which further provides even flow distribution and mixing into microarray 300. A plurality of microposts 332 also help support a cover substrate (not shown). Fluid entering the microarray 300 through the sixteen total branches of micromanifold 321 enter micropost region 33 1. Fluid subsequently flows into micropost region 330 and subsequently out of the microarray region through micromanifold 320, which then exits the microfluidic chip through microchannel 94. An observation port 400 of approximate dimensions 16 millimeters by 16 millimeters is provided on a cover substrate, which is typically situated above the microarray region 300, but the observation port can be situated below the microarray too.

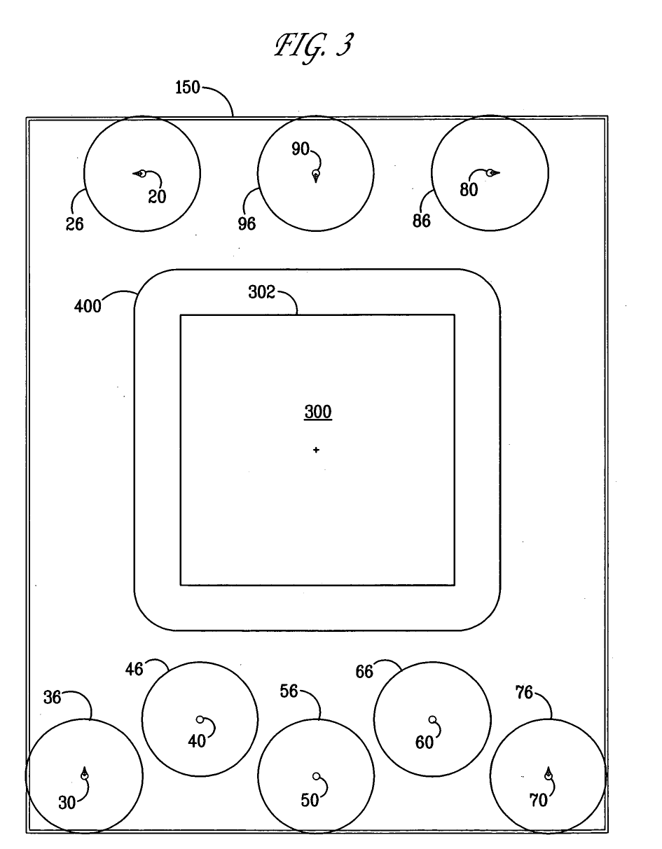

FIG. 3 depicts a cover substrate 150 having vias 20, 30, 40, 50, 60, 70, 80, and 90 surrounded by circular regions 26, 36, 46, 56, 66, 76, 86 and 96 for connection to external fluidic structures (not shown). Also depicted is the observation port 400 situated around the microarray region 302. During use in a microanalytical device, the observation port is viewably positioned with a suitable microarray detector. Examples of suitable microarray detectors include a conventional scanner, such as the “Typhoon”™ device available from Amersham Biosciences, a microscope, or a microarray detector that is disclosed in U.S. Pat. No. 6,567,163 “Microarray Detector and Synthesizer” to Sandstrom, the entire contents of which are incorporated by reference thereto. The observation port can include the cover substrate material contained within region 400, or the substrate material can be at least partially removed, or completely removed. The observation port is preferably provided with the substrate material completely removed to enable the spotting of the microarray probes after the cover and base substrates are sealed.

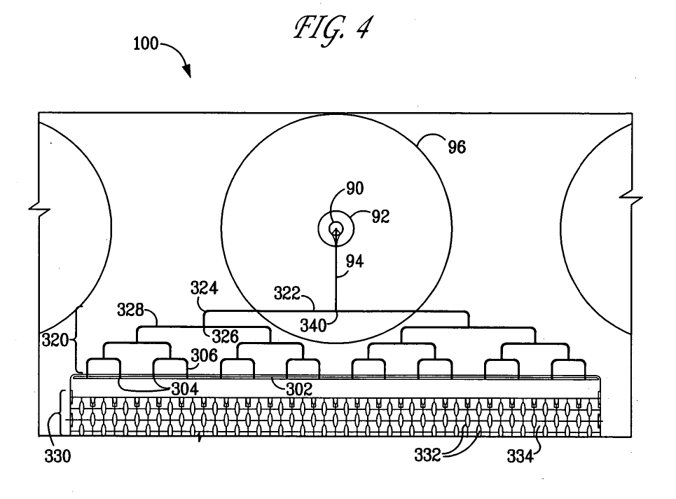

FIG. 4 depicts a magnified portion of the microfluidic chip of FIG. 2. Shown in greater detail is via 90 surrounded by circular regions 92 and 96 for, respectively, an O-ring and for connection to external fluidic structures, such as a waste vial. Via 90 is in fluidic communication with microchannel 94, which is in fluidic communication with micromanifold 320 at junction 340. The micromanifold 320 includes four series of branches, each series having two branches for a total of sixteen branches 306. The first series is depicted having a branch 322 and a bend that terminates at junction 326. Branch 328 represents a portion of the second series, and so on. The sixteen branches 306 of the fourth series are shown in fluidic communication with the microarray at the microarray wall 302 through openings 304. Micropost array 330 includes a series of overlapping alternating rows of a plurality of microposts 332 separated by regions 334. The micropost array and the micromanifold is designed to provide even fluid flow and enhanced mixing between the via 90 and the microarray 300 (not shown).

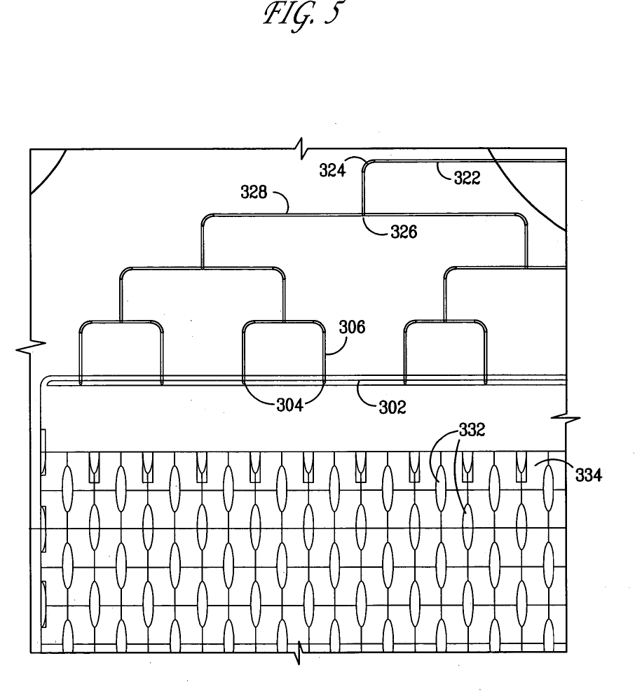

FIG. 5 depicts a further magnified portion of the microfluidic chip of FIG. 4.

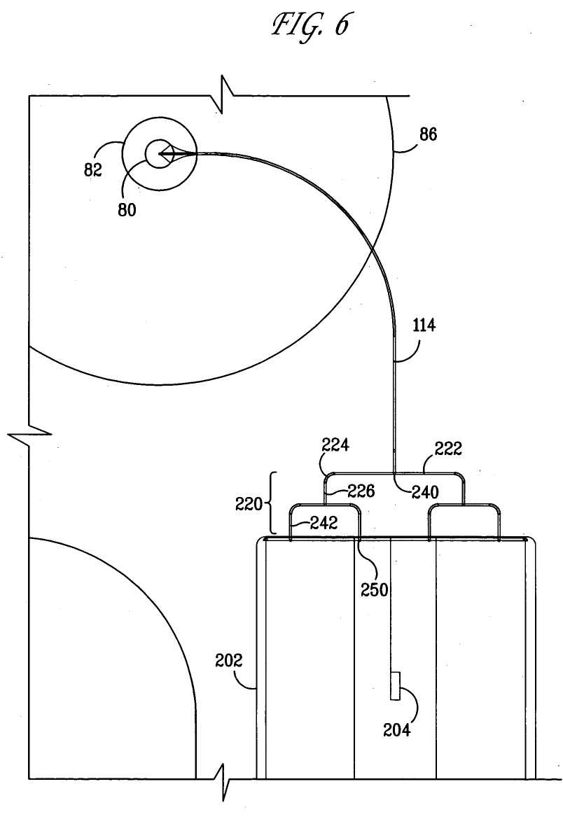

FIG. 6 depicts a magnified portion of the microfluidic chip of FIG. 2. Shown in greater detail is via 80 surrounded by circular regions 82 and 86 for, respectively, an O-ring and for connection to external fluidic structures, such as a sample injector. Microchannel 114 fluidically connects via 80 to the reservoir 200 through micromanifold 220. Microchannel 114 truncates at junction 240, which forms the first series of two branches 222 and 226, each having a bend 224. The second series of branches, one of which is demarcated 242, is shown entering reservoir 200 at openings 250. Support post 204 is also shown. Not shown is functionalized porous polymer monolith within reservoir 202.

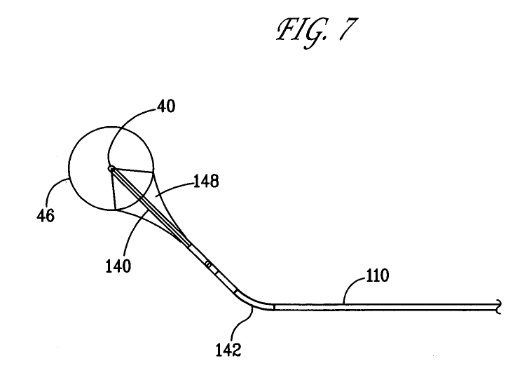

FIG. 7 depicts a magnified portion of the microfluidic chip of FIG. 2. Shown in greater detail is via 40 surrounded by circular region 86 for placement of an O-ring for connection to external fluidic structures, such as a target injector. Microchannel 110 fluidically connects via 40 through a bend 142 connected to portion 140. A kite-shaped region 148 along with microchannel 140 provides for a gradual fluid flow transition between via 40 and microchannel 110.

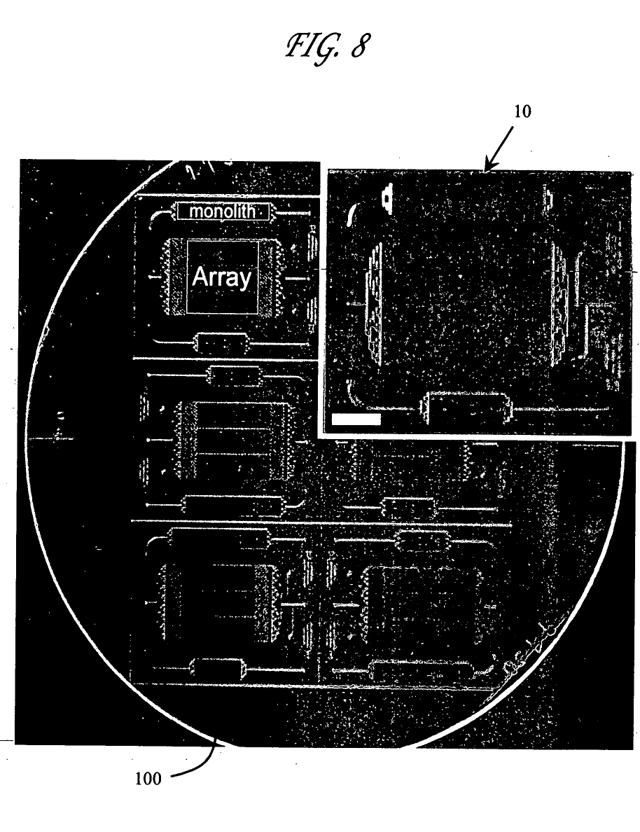

FIG. 8 depicts a base substrate 100 having six microfluidic chips 10 etched therein. The two by three arrangement of six microfluidic chips is prepared on a 100 millimeter fused silica substrate. Placement of the functionalized porous polymer monolith (marked “monolith”) and the microarray (marked “array”) are preferably indicated.

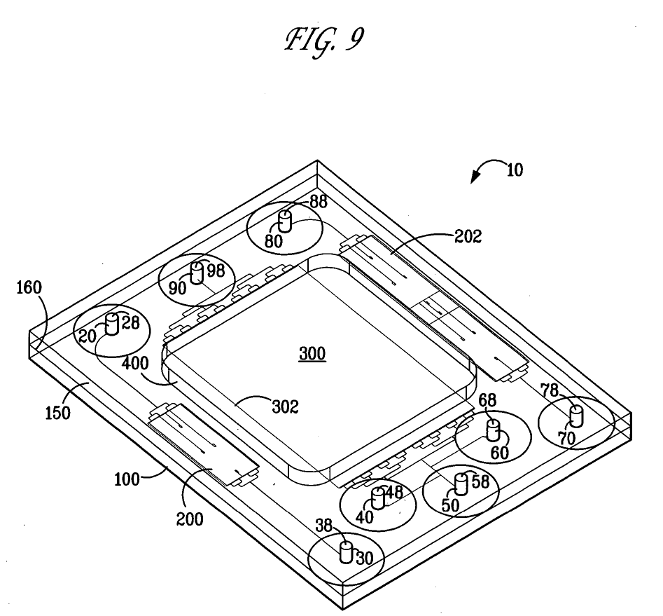

FIG. 9 is a perspective view of another embodiment of a microfluidic chip of the present invention. Base substrate 100 is shown bonded to cover substrate at bonding plane 160. The microarray region 300 is provided in the bonding plane, which is situated on the base substrate 100. Access to the gene spotting area of the microarray region is through the observation port 400, which is provided as a missing portion of the cover substrate. The eight vias 20, 30, 40, 50, 60, 70, 80 and 90 are shown formed in the cover substrate, each having an opening in the top surface of the cover substrate, 28, 38, 48, 58, 68, 78, 88 and 98 respectively, for transportation of fluids with fluid structures external to the microfluidic chip. The microchannels in fluidic communication with the eight vias, and the reservoirs 200 and 202 are shown as microstructures etched in the base substrate 100 and sealed at the bonding layer 160 to cover substrate 150: The functionalized porous polymer monolith (not shown) is typically polymerized in place in reservoir 202.

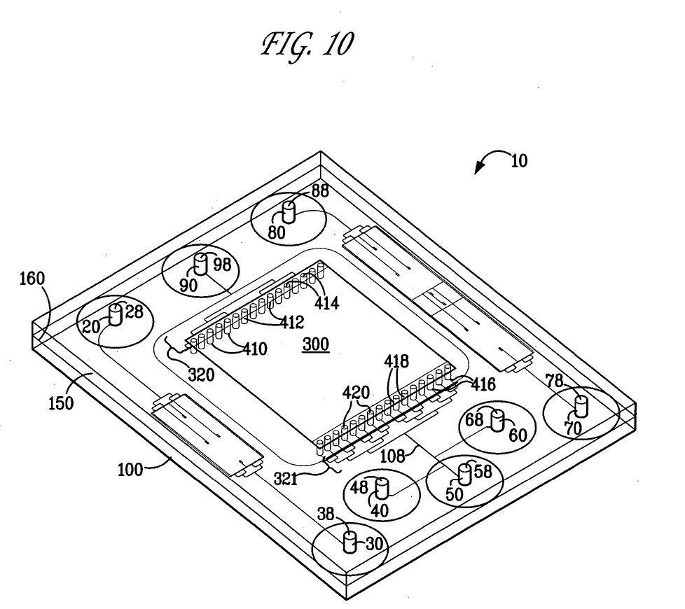

FIG. 10 is a perspective view of one embodiment of the microfluidic chip of the present invention. This embodiment is similar to that shown in FIG. 9 with the additional features of the microarray 300 being in fluidic communication with a plurality of vias 412 and 418 through openings 414 and 420 respectively. The plurality of vias 412 and 418 are further in fluidic communication with micromanifolds 320 and 321 through openings 410 and 416, respectively. The microarray region 300 is open and resides on top of the cover substrate 150.

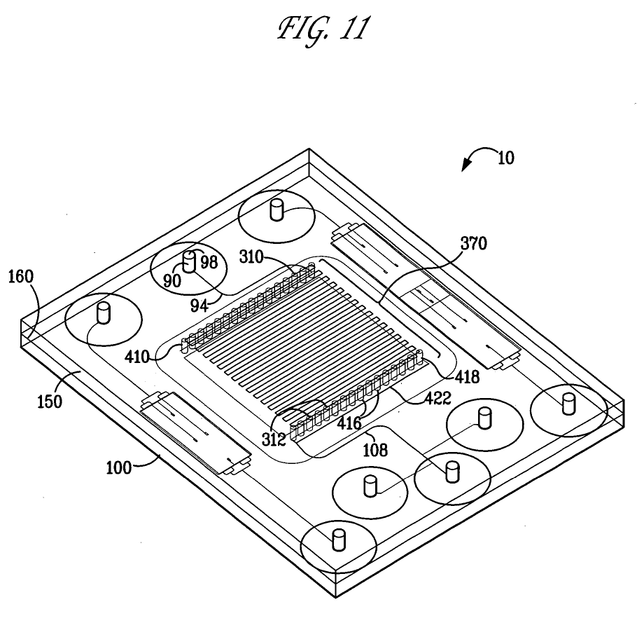

FIG. 11 is a perspective view of one embodiment of the microfluidic chip of the present invention. This embodiment is similar to that shown in FIG. 10 with an additional feature of the microarray region being arranged in a serpentine fashion on the top surface of cover substrate 150. The serpentine microarray region includes a plurality of linear microchannel segments 370 etched in the cover substrate surface that are connected by a plurality of via junctions (not shown) to provide a continuous one-dimensional type serpentine fluid path between vias 310 and 312. The microarray region is open and resides on top of the cover substrate 150. The probes (not shown) can be placed directly into the serpentine microchannel. Fluid enters the serpentine microchannel from plurality of vias 418 that are in fluidic communication with microchannel 422 through openings 416. Microchannel 422 receives fluid (such as target molecules in buffer solution, not shown) from microchannel 108. During operation, the serpentine microchannels are sealed using a suitable detector window or substrate (not shown) to enable fluid transport from serpentine channel entrance 312 to serpentine microchannel exit 310. Fluid exits the serpentine microchannel at exit via 310 and enters microchannel 94. Fluid exits the microfluidic chip 10 from via 90 by way of via opening 98.

FIG. 12 shows another embodiment of the microfluidic chip of the present invention. In this embodiment, microfluidic chip 10 includes structures similar to those depicted in FIG. 1, and further contains micromanifolds 220 for the two reservoirs, reservoir 200 being a smaller reservoir of approximate dimensions 3 millimeters by 8 millimeters and reservoir 202 being a larger reservoir of approximate dimensions 3 millimeters by 16 millimeters. Also, the microarray region 302 is arranged in a serpentine fashion as a plurality of interconnected linear microchannel segments 370 on the top surface of cover substrate 150 (not shown). In an alternative embodiment, the microchannel segments can be etched in the base substrate 100. The arrangement of the microchannel segments of the serpentine microarray is approximately 12 millimeters by 12 millimeters, which includes a plurality of linear microchannel segments 370 connected by a plurality of via junctions 374 to provide a continuous linear type serpentine fluid path of approximately 350 millimeters long between vias 310 and 312. Each of the microchannel segments 370 is approximately 12 millimeters long, 300 microns wide, and 10 microns deep. A plurality of walls 372, approximately 100 microns thick, separate each of the microchannel segments 370. The microchannel segments of the serpentine microarray region are open and reside on top of the cover substrate 150 (not shown). The probes (not shown) can be placed directly into the serpentine microchannel. The substrate 100 in this embodiment is approximately 25 millimeters wide by 32 millimeters long by about 1 millimeter thick. The ten vias 20, 30, 40, 50, 60, 70, 80, 90, 310 and 312 are approximately 300 microns wide. In an alternate embodiment where the microchannel segments 370 are provided in the base substrate, vias 310 and 312 are typically absent to provide fluidic communication directly between microchannels 94 and 108 with the microchannel segments 370. The microchannels have a depth between about 10 and 40 microns. During operation, fluids are transported through microchannel 108 into via 312, which leads to the serpentine microarray region on the surface of cover substrate 150 (not shown). Fluid subsequently flows in a serpentine fashion through each of the plurality of microchannel segments 370 by way of via junction 374, and out of the microarray region through microarray exit via 310, which then exits the microfluidic chip through microchannel 94 and waste via 90. A squarish observation port 400 of approximate dimensions 16 millimeters by 16 millimeters is provided on a cover substrate, which is typically situated above the microarray region. Alternatively or in addition, the observation port 400 can be situated below the microarray. During operation a squarish O-ring seal (not shown) is provided around the perimeter of the observation port to seal the open surface of the probe-spotted microarray region to a suitable detector substrate or window. The O-ring seal and suitable detector substrate or window contains the fluid flow within the microarray.

FIG. 13 shows another embodiment of the microfluidic chip of the present invention. In this embodiment, microfluidic chip 10 includes structures similar to those depicted in FIG. 12, with the exception that the serpentine microarray region is contained within a circular region, denoted a “circular serpentine” path. Total microarray path length is approximately 343 millimeters. The circular serpentine microarray region has a diameter of approximately 14 millimeters and is sealed to a suitable detector substrate or window using a circular O-ring of slightly larger diameter (not shown) that resides within region 314. The microfluidic chip depicted in FIG. 13 is preferred over that depicted in FIG. 12 as the circular O-ring seals more easily than the squarish O-ring.

Examples of methods of fabricating microfluidic systems is known, as disclosed in U.S. Pat. No. 5,194,133 to Clark et al., U.S. Pat. No. 5,132,012 to Miura et al., U.S. Pat. No. 4,908,112 to Pace, U.S. Pat. No. 5,571,410 to Swedberg et al., U.S. Pat. No. 5,824,204 to Jerman, and U.S. Patent Application Pub. No. 2002/194,909 to Shepodd et al., the disclosures of each pertaining to the fabricating of microfluidic systems is incorporated by reference thereto.

### EXAMPLES

A microfluidic chip was fabricated containing a DNA gene microarray that is capable of detecting thousands of genes using a single experimental sample. The microfluidic chip of FIG. 8, inset, was produced in fused silica using standard wet-etching photolithography procedures. The chip layout is essentially the same as that depicted in FIG. 2. The compact design feature (2.5 cm×3.1 cm) enabled the production of six devices per wafer and enabled the use of low volumes of fluids suitable for gene microarray analysis. This microfluidic chip design also provided the ability to spot content genes after thermal bonding of the base and cover substrates (1100° C.). The open microarray probe design provided the ability to rapidly change the probe content sets. The open microarray design also enabled the performance of custom cDNA microarray construction (up to approximately 14000 spots) using a custom fabricated arrayer which can be programmed to deposit probes in complex architectures (e.g., the one described above by Schena and Brown). The integration of the microfluidic channels (approximately 100 microns wide) with the relatively large surface area microarray (1.2 square centimeters) was accomplished using a sixteen total branch (four series/two branches per series) manifold design that utilized several channel depths to insure maximal sample mixing.

Functionalized porous polymer monoliths were prepared in a reservoir to capture and concentrate target genes (mRNA). An ultraviolet-light initiated porous polymer monolith precursor was polymerized within the reservoir according to the following method: First the microfluidic chip was pretreated with a ethoxysilane molecule to facilitate polymer binding to the channel wall. This step was performed by passing an acidic solution containing 10 parts water, 6 parts acetic acid, and 4 parts z-6030 through the fluidic channel for a period of three hours. The chip was then flushed with water for a period of thirty minutes. The monomer solution which contains 1940 uL methanol, 660 uL ethyl acetate, 840 uL GMA, 560 uL EGDMA, and 8 mg of Irgacure™ 1800 was first degassed by either sonication and then introduced to the channel at room temperature. Photo-initiation of the polymer was performed using a UV crosslinking oven set at 350 nm. The polymerization of the monolith proceeded for a period between 10 seconds to 30 minutes. After polymerization was complete, the channel was flushed with a solution of acetonitrile and water for a period of three hours at a flow rate of 1 ul/min. When flushing was completed, a solution consisting of oligo(dT) which contains a terminal free amine, sodium phosphate, acetonitrile and water was introduced into the porous polymer monolith. This solution was allowed to react with the monolith for a period no less than about one hour and no more than about 48 hours. When functionalization was complete the channel was flushed with an acetonitrile and water solution. The porous polymer monolith was post-functionalized with poly-A, an amine-containing oligonucleotide for the capture of mRNA target genes.

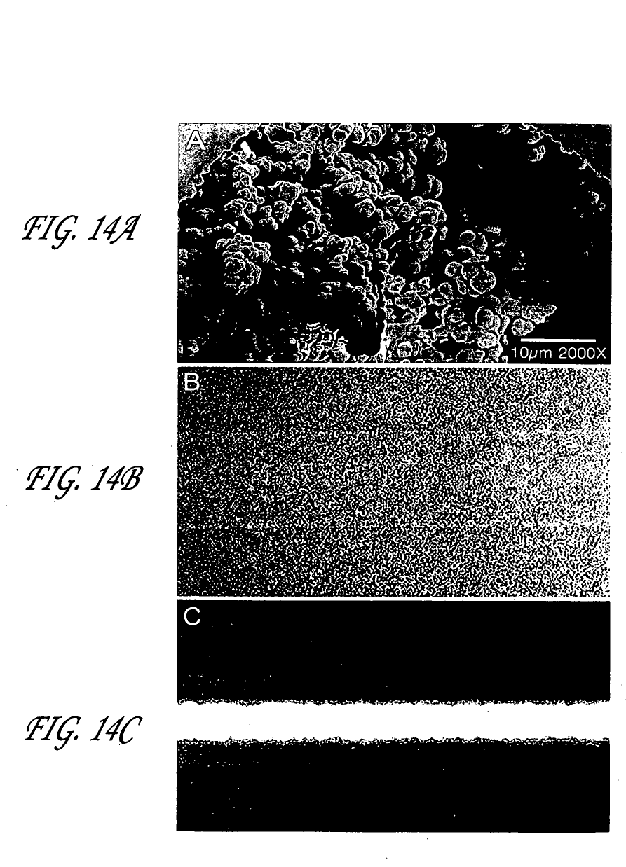

FIG. 14-A shows a scanning electron microscope picture of the porous polymer monolith, as described in the previous paragraph, prior to functionalization. FIGS. 14-B and 14-C are micrographs of the porous polymer monolith within a capillary taken with a fluorescent microscope at 488 nanometers. FIG. 14-B is an image of the polymerized monolith before it is functionalized, which does not fluoresce when illuminated under 488 nm wavelength light. FIG. 14-C is an image after the monolith is functionalized with an oligonucleotide poly (dT) that contains a fluorescent dye molecule with a functional amine. This monolith shows a high degree of fluorescence on the functionalized porous monolith, which indicates that a high degree of post-functionalization can be achieved using glycidyl functionalized monolith. Accordingly, these monoliths are useful for trapping and concentrating complementary oligonucleotides such as mRNA (i.e. binding by hybridization to the oligo-T functional groups). Unlike many polymer gels, the functionalized porous polymer monolith does not require frits to be contained within the reservoir when operated under pressure. Porosity of the functionalized porous polymer monolith was suitably high to maintain the operating pressure below about 1000 PSI.

The microarray is prepared by robotically depositing oligonucleotides on the array surface. The arraying surface is prepared for oligo deposition as follows. First, the arraying area is coated with a thin chemical monolayer which allows deposited oligonucleotides to covalently react with the arraying surface to provide a stable oligonucleotide deposition. For example, the surface of the array is exposed to concentrations of 0.5-10% trimethoxysilane aldehyde in an acidic hexane/water solution. After the surface is prepared, spotting is carried out using an arrayer that has the ability to spot in complex architectures, such as deep microfluidic wells or serpentine channels, such as the one described above by Schena and Brown. After deposition the slides are washed with sodium citrate solution to remove extraneous fluorescent compounds and unbound oligonucleotides.

When the microfluidic chip has both oligo (dT) functionalized porous polymer monolith and spotted microarray, the device is assembled into a manifold that can accommodate all via holes and perform detection of the array. First, a sample containing mRNA is infused into the monolith channel in a high salt solution, at about 25° C. The sample is then washed with a high salt containing buffer, then a low salt containing buffer to remove unwanted interferents, such as genomic DNA and protein which may not be of interest. After the sample is washed, a reactive dye is introduced into the channel to label the trapped oligonucleotides with a fluorescent dye. Once the free dye has been removed from the monolith channel the microfluidic chip is heated to about 95° C. This elevated temperature releases the labeled samples from the trapping monolith and prepares the array surface for oligonucleotide hybridization on the array. The labeled target sample is then moved on to the array surface where it binds or hybridizes with oligonucleotide probes. During this movement the array is cooled to 55° C., which allows the fluorescently tagged target analytes to hybridize with the probes spotted on the array. This hybridization results in the detection of one or several 120 um fluorescent spots which corresponds to one of the spotted microarray probes. These probes are spatially indexed to provide the ability to identify each probe located on the array. One or several of these probes may be detected in any given sample. A specific combination of the detected probes corresponds to a particular organism, and a complex combination can identify complex mixtures of organisms, or identify organisms in a complex environmental background, or both.

1. A microfluidic chip, comprising:
a plurality of vias;
a functionalized porous polymer monolith capable of being in fluid communication with at least one of said vias;
a microarray capable of being in fluid communication with said functionalized porous polymer monolith; and
an observation port through which at least one target disposed within said microarray is capable of being detected.

2. The microfluidic chip of claim 1, wherein said microarray comprises at least one probe.

3. The microfluidic chip of claim 2, wherein said probe is capable of binding said at least one target.

4. The microfluidic chip of claim 3, wherein said at least one target comprises a nucleic acid, a protein, an antigen, an antibody, or any combination thereof.

5. The microfluidic chip of claim 4, wherein said nucleic acid comprises RNA, DNA, LNA, PNA, HNA, or any combination thereof.

6. The microfluidic chip of claim 4, wherein said nucleic acid of said target is capable of hybridizing with a nucleic acid of said probe.

7. The microfluidic chip of claim 6, wherein said nucleic acid of said target comprises cDNA.

8. The microfluidic chip of claim 4, wherein said nucleic acid comprises an oligonucleotide.

9. The microfluidic chip of claim 4, wherein said nucleic acid comprises a single stranded nucleic acid, a double stranded nucleic acid, or any combination thereof.

10. The microfluidic chip of claim 5, wherein said DNA is cDNA.

11. The microfluidic chip of claim 3, wherein said microarray comprises a plurality of probes capable of binding a plurality of targets.

12. The microfluidic chip of claim 11, wherein said microarray comprises at least about 1,000 probes.

13. The microfluidic chip of claim 11, wherein said microarray comprises at least about 5,000 probes.

14. The microfluidic chip of claim 11, wherein said microarray comprises at least about 10,000 probes.

15. The microfluidic chip of claim 11, wherein said microarray comprises up to about 50,000 probes.

16. The microfluidic chip of claim 11, wherein said plurality of probes comprises at least one probe different than the other probes.

17. The microfluidic chip of claim 16, wherein said probe different than the other probes is capable of binding at least one target different than the other targets.

18. The microfluidic chip of claim 17, wherein each of said probes is individually capable of binding a target different than the other targets.

19. The microfluidic chip of claim 2, wherein said at least one probe is disposed as at least one spot on the surface of a base substrate.

20. The microfluidic chip of claim 19, wherein said at least one spot is at least about 10 microns wide.

21. The microfluidic chip of claim 19, wherein said at least one spot is at least about 20 microns wide.

22. The microfluidic chip of claim 19, wherein said at least one spot is at least about 40 microns wide.

23. The microfluidic chip of claim 19, wherein said at least one spot is at least about 60 microns wide.

24. The microfluidic chip of claim 19, wherein said at least one spot is at most about 250 microns wide.

25. The microfluidic chip of claim 11, wherein said plurality of probes are disposed as a plurality of spots on the surface of a base substrate.

26. The microfluidic chip of claim 25, wherein said plurality of spots are each separated from one another by at least about 10 microns.

27. The microfluidic chip of claim 25, wherein said plurality of spots are each separated from one another by at least about 20 microns.

28. The microfluidic chip of claim 25, wherein said plurality of spots are each separated from one another by at least about 50 microns.

29. The microfluidic chip of claim 25, wherein said plurality of spots are each separated from one another by at least about 100 microns.

30. The microfluidic chip of claim 25, wherein said plurality of spots are each separated from one another by at most about 500 microns.

31. The microfluidic chip of claim 11, wherein said plurality of probes comprise an ordered arrangement.

32. The microfluidic chip of claim 31, wherein said ordered arrangement comprises from one to three dimensions.

33. The microfluidic chip of claim 31, wherein said plurality of probes are linearly arranged.

34. The microfluidic chip of claim 31, wherein said plurality of probes are linearly arranged in two dimensions.

35. The microfluidic chip of claim 33, wherein said plurality of probes are disposed as a plurality of spots on the surface of a base substrate.

36. The microfluidic chip of claim 35, wherein said plurality of spots are disposed within at least one microchannel.

37. The microfluidic chip of claim 36, wherein said at least one microchannel varies in direction along said surface of the substrate.

38. The microfluidic chip of claim 37, wherein said microchannel is from about 10 microns to about 500 microns wide and from about 1,000 microns to about 1,000,000 microns long.

39. The microfluidic chip of claim 37, wherein said at least one microchannel is disposed as a spiral path, a serpentine path, a curved path, a straight path in fluid communication with at least one other path, or any combination thereof.

40. The microfluidic chip of claim 39, wherein said serpentine path comprises a circular serpentine path, a rectangular serpentine path, or any combination thereof.

41. The microfluidic chip of claim 40, wherein a first section of said serpentine path is disposed adjacent to a second section of said serpentine path, the first and second sections being separated by a wall of non-zero thickness.

42. The microfluidic chip of claim 41, wherein the thickness of said wall is in the range of from about 10 microns to about 1,000 microns.

43. The microfluidic chip of claim 31, wherein said plurality of probes are planarly arranged in two dimensions.

44. The microfluidic chip of claim 43, wherein said plurality of probes are disposed as a plurality of spots on the surface of a base substrate.

45. The microfluidic chip of claim 44, wherein the plurality of spots are arranged in rows and columns, said rows and columns each numbering from about 10 to about 1,000.

46. The microfluidic chip of claim 44, wherein said plurality of spots are disposed within a microwell.

47. The microfluidic chip of claim 31, wherein said plurality of probes are spatially arranged in three dimensions.

48. The microfluidic chip of claim 11, wherein said plurality of probes comprise a disordered arrangement.

49. The microfluidic chip of claim 48, wherein said plurality of probes are disposed as a plurality of spots on the surface of a base substrate.

50. The microfluidic chip of claim 49, wherein the mean distance between the plurality of spots is in the range of from about 10 to 500 microns.

51. The microfluidic chip of claim 19, wherein said probe is capable of binding said at least one target, and said spot comprises at least about one monolayer of said probe.

52. The microfluidic chip of claim 51, wherein said probes comprise nucleic acids capable of hybridizing with said at least one target.

53. The microfluidic chip of claim 19, wherein said at least one probe is covalently bonded to said substrate.

54. The microfluidic chip of claim 53, further comprising a linker molecular covalently bonded between said probe and said substrate.

55. The microfluidic chip of claim 11, wherein said plurality of probes comprise nucleic acids, proteins, antigens, antibodies, or any combination thereof.

56. The microfluidic chip of claim 55, wherein said nucleic acids comprise RNA, DNA, LNA, PNA, HNA, or any combination thereof.

57. The microfluidic chip of claim 55, wherein said nucleic acids comprise an oligomer.

58. The microfluidic chip of claim 55, wherein said nucleic acids comprise a single stranded nucleic acid, a double stranded nucleic acid, or any combination thereof.

59. The microfluidic chip of claim 56, wherein said nucleic acids comprise cDNA.

60. The microfluidic chip of claim 59, wherein said target comprises cDNA capable of hybridizing with said probe.

61. The microfluidic chip of claim 1, wherein said functionalized porous polymer monolith is capable of binding a nucleic acid.

62. The microfluidic chip of claim 1, wherein said functionalized porous polymer monolith comprises pores having a surface, said pores permitting fluid communication through said functionalized porous polymer monolith.

63. The microfluidic chip of claim 62, wherein said functionalized porous polymer monolith comprises a highly crosslinked polymer.

64. The microfluidic chip of claim 63, wherein said highly crosslinked polymer comprises units derived from at least one mono-ethylenically unsaturated monomer, at least one multi-ethylenically unsaturated monomer, or a combination thereof.

65. The microfluidic chip of claim 64, wherein said at least one mono-ethylenically unsaturated monomer comprises glycidyl methacrylate.

66. The microfluidic chip of claim 64, wherein said at least one multi-ethylenically unsaturated monomer comprises ethylene glycol dimethacrylate.

67. The microfluidic chip of claim 63, wherein said highly crosslinked polymer comprises units derived from a radical reaction catalyzed by UV activation of bis(2,6-D, methoxybenzoyl)-2,4,4-trimethylphenyl phosphine oxide.

68. The microfluidic chip of claim 62, wherein said functionalized porous polymer monolith comprises pores smaller than about 10 microns.

69. The microfluidic chip of claim 62, wherein said functionalized porous polymer monolith comprises a void fraction of less than about 50 percent based on volume of said functionalized porous polymer monolith.

70. The microfluidic chip of claim 62, wherein said functionalized porous polymer monolith is capable of operating at pressures between 100 and 3000 PSI in an aqueous fluid at 25° C. that is communicated therethrough.

71. The microfluidic chip of claim 62, wherein said functionalized porous polymer monolith is covalently bonded to a substrate.

72. The microfluidic chip of claim 62, wherein said functionalized porous polymer monolith comprises at least one functional group for binding a sample compound.

73. The microfluidic chip of claim 72, wherein said functional group comprises an amine-containing ligand, an alcohol-containing ligand, a thiol-containing ligand or a hydrazine-containing ligand, or any combination thereof.

74. The microfluidic chip of claim 72, wherein said functional group comprises a nucleic acid, a protein, an antibody, an antigen, an amine-containing ligand, or any combination thereof.

75. The microfluidic chip of claim 74, wherein said nucleic acid comprises an oligonucleotide.

76. The microfluidic chip of claim 75, wherein said oligonucleotide comprises oligo-T.

77. The microfluidic chip of claim 75, wherein said at least one target comprises cDNA capable of binding at least a portion of said oligonucleotide.

78. The microfluidic chip of claim 1, wherein said microarray and said functionalized porous polymer monolith are disposed between a base substrate and a cover substrate.

79. The microfluidic chip of claim 78, wherein said microarray is disposed on a top surface of said cover substrate.

80. The microfluidic chip of claim 79, wherein said cover substrate comprises a region above said microarray to provide said observation port.

81. The microfluidic chip of claim 1, wherein both of said functionalized porous polymer monolith and said microarray are disposed between a base substrate and a cover substrate.

82. The microfluidic chip of claim 81, wherein said plurality of vias are disposed within said base substrate, said cover substrate, or any combination thereof, said vias being in fluid communication with said functionalized porous polymer monolith, said microarray, or both.

83. The microfluidic chip of claim 82, wherein said vias are capable of being in fluid communication with fluidic devices external to said microfluidic chip.

84. The microfluidic chip of claim 1, wherein at least one of said vias is not in fluid communication with said functionalized porous polymer monolith.

85. The microfluidic chip of claim 1, wherein said functionalized porous polymer monolith is not in fluid communication With said microarray.

86. The microfluidic chip of claim 81, wherein said base substrate and said cover substrate are at least partially bonded together at a bonding surface.

87. The microfluidic chip of claim 86, wherein said base substrate comprises at least one microfluidic structure disposed at said-bonding surface.

88. The microfluidic chip of claim 87, wherein said microfluidic structure comprises a microchannel, a microwell, a reservoir, a microelectrode, a microjunction, a microsplitter, a microfilter, a microreactor, a microvalve, a microsensor, a microinjector, a micromixer, a micropump, a microseparator, a micromanifold, or any combination thereof.

89. The microfluidic chip of claim 87, wherein said functionalized porous polymer monolith is disposed within said microfluidic structure.

90. The microfluidic chip of claim 89, wherein said microfluidic structure comprises a microchannel, a microwell, a reservoir, or any combination thereof.

91. The microfluidic chip of claim 89, wherein said microfluidic structure further comprises microposts bonded between said base substrate and said cover substrate, said microposts being capable of reducing the deformation of said cover substrate disposed above said microfluidic structure, being capable of mixing fluid flowing through said microfluidic structure, or both.

92. The microfluidic chip of claim 90, wherein said microwell or reservoir further comprises a micromanifold, said micromanifold capable of equalizing the pressure distribution within said microfluidic structure.

93. The microfluidic chip of claim 87, wherein said microarray is disposed within said microfluidic structure.

94. The microfluidic chip of claim 93, wherein said microfluidic structure comprises a microchannel, a microwell, a reservoir, or any combination thereof.

95. The microfluidic chip of claim 93, wherein said microwell or reservoir further comprises a micromanifold, said micromanifold capable of equalizing the pressure distribution within said microfluidic structure.

96. The microfluidic chip of claim 94, further comprising a microfluidic injector in fluid communication with said microfluidic structure, said microfluidic injector being capable of providing a fluid plug into said microarray.

97. The microfluidic chip of claim 96, wherein said microfluidic structure comprises a microwell or reservoir, and said microfluidic chip further comprising a microchannel disposed between said microfluidic injector and said microwell or reservoir.

98. The microfluidic chip of claim 87, wherein said microfluidic structure is disposed in a region comprising a dimension perpendicular to said bonding surface, said dimension being up to about 1,000 microns.

99. The microfluidic chip of claim 98, wherein said dimension is in the range of from about 1 to about 500 microns.

100. The microfluidic chip of claim 98, wherein said dimension is in the range of from about 5 to about 250 microns.

101. The microfluidic chip of claim 98, wherein said dimension is in the range of from about 10 to about 100 microns.

102. The microfluidic chip of claim 87, wherein said microfluidic structure is disposed in a region comprising a dimension parallel to said bonding surface, said dimension being up to about 100,000 microns.

103. The microfluidic chip of claim 102, wherein said dimension is in the range of from about 10 to about 50,000 microns.

104. The microfluidic chip of claim 102, wherein said dimension is in the range of from about 50 to about 25,000 microns.

105. The microfluidic chip of claim 102, wherein said dimension is in the range of from about 100 to about 10,000 microns.

106. The microfluidic chip of claim 86, wherein said base substrate comprises a plurality of microfluidic structures in said bonding surface.

107. The microfluidic chip of claim 106, wherein said plurality of microfluidic structures comprises a microchannel, a microwell, a reservoir, a microelectrode, a monolith channel, or any combination thereof.

108. The microfluidic chip of claim 86, wherein said cover substrate comprises a region not bonded to said base substrate to provide said observation port.

109. The microfluidic chip of claim 108, wherein said region comprises an opening in said cover substrate.

110. The microfluidic chip of claim 109, wherein said opening is disposed above said microarray.

111. The microfluidic chip of claim 1, further comprising a derivatization reservoir capable of being in fluid communication with said functionalized porous polymer monolith.

112. The microfluidic chip of claim 111, wherein said derivatization reservoir comprises a functionalized porous polymer monolith for trapping target nucleic acids.

113. The microfluidic chip of claim 111, wherein said derivatization reservoir comprises an oligo (dT), a random oligo sequence, a gene family specific sequence, a protein ligand, or a protein receptor, or any combination thereof for stabilizing target nucleic acids or proteins.

114. The microfluidic clip of claim 1, further comprising one or more mobile monolith valves capable of controlling fluid flow in said microfluidic chip.
